(19) United States
(12) Patent Application Publication
(10) Pub. No.: US 2009/0235930 A1
(43) Pub. Date: Sep. 24, 2009

(54) UNIT DOSE DRY POWDER INHALER

(76) Inventors: Matthew E. Young, Cambridge (GB); Stuart Brian William Kay, Cambridge (GB); Neil Richard Harrison, Birmingham (GB); Michael W. Ligotke, San Diego, CA (US)

Correspondence Address: PERKINS COIE LLP, POST OFFICE BOX 1208, SEATTLE, WA, 98111-1208, US

(21) Appl. No.: 12/476,985

(22) Filed: Jun. 2, 2009

Related U.S. Application Data
(63) Continuation of application No. 11/130,959, filed on May 17, 2005, now abandoned.
(60) Provisional application No. 60/575,138, filed on May 28, 2004.

(51) Int. Cl. A61M 15/00; A61M 16/10

(52) U.S. Cl. 128/203.15

(57) ABSTRACT

A unit dose dry powder inhaler has a dose unit within an inhaler housing. As the dose unit is pushed into the inhaler housing, a container on the dose unit is opened, releasing a pharmaceutical powder into the housing. The user inhales on a mouthpiece, drawing the powder and air into a dispersion chamber. The powder disperses in air, and the powder/air mixture is inhaled. The inhaler is inexpensive, compact, and simple to use.

This application is a Continuation of U.S. patent application Ser. No. 11/130,959, filed May 17, 2005, and now pending, which claims priority to U.S. Provisional Application No. 60/575,138 filed May 28, 2004.

### BACKGROUND OF INVENTION

The field of the invention is dry powder inhalers.

Certain medicines may be inhaled in dry powder form directly into the lungs. Inhalation bypasses the digestive system and avoids any potential metabolic inactivation or destruction of the medicine by the digestive system. Inhalation can also provide very rapid onset of the effect of the medicine. Inhalation may also allow smaller doses to be used to achieve the same desired results as orally ingested medicines. In other cases, it provides a delivery technique for medicines that display unacceptable side effects when taken by other methods. In addition, inhalation also avoids the potential risks of injection to both medical care givers and patients.

Various inhaler designs have been proposed, to allow dry powder medicines to be inhaled. Most of these inhalers are metered dose inhalers or multiple dose dry powder inhalers. Metered dose inhalers dispense a suspension of powder particles in a compressed propellant gas. Multiple dose dry powder inhalers generally repeatedly dispense individual doses from a bulk powder reservoir, or from a blister disk, cassette or tape. However, certain medicines, such as certain peptides or proteins, or medicines such as vaccines, antidotes, etc., are generally taken by a patient infrequently or may even be taken only once. Metered dose inhalers and multiple dose dry powder inhalers are not intended or well designed for one-time use, to deliver a single dose. These types of inhalers are typically too bulky, costly, inefficient, or difficult to use, when only a single dose is desired, and where the inhaler can be and its drug/dose unit practically discarded after use, in an environmentally acceptable way.

Several unit dose inhalers, intended for one-time use, have been proposed. However, they have not achieved widespread use. Disadvantages remain with unit dose inhalers relating to powder storage, dose uniformity, dispersion performance, ease of use, cost, and other factors. Accordingly, there is a need for an improved inhaler for efficiently providing a single dose of a powdered drug.

It is an object of the invention to provide such an improved dry powder inhaler.

### SUMMARY OF THE INVENTION

A unit dose dry powder inhaler uses a dose unit containing a single dose of a pharmaceutical powder in a blister or other sealed container. As the dose unit is moved into the inhaler, the container is automatically opened, releasing the dose of powder for inhalation. This inhaler design is simple, compact, inexpensive and yet efficient.

In one aspect, the inhaler has a dispersion chamber including one or more beads. Upon inhalation, the beads move rapidly within the dispersion chamber to help disperse the powder into air. Accordingly, the powder is effectively dispersed via air flow resulting from inspiration of the user. While preferred, the beads are not an essential element.

In another aspect, a dose unit or assembly has a tab pivotably supported within the plane of a frame. The tab is separated from the frame by a slot. A container, such as a blister containing a single dose of powder medicine has a bottom surface or base layer attached to the tab. The base layer also extends over the slot and is attached to areas of the frame. As dose unit is moved typically linearly, within the inhaler, the tab pivots out of the plane of the frame. This shears or tears open the base layer releasing the powder into the inhaler. As a result, the dose of powder remains sealed within the blister or container, until moments just before inhalation. The disadvantages of prolonged exposure of the powder to the environment, such as oxidation, particle size growth, caking, etc. are reduced or avoided. In addition, the inhaler with the dose unit is very easy to use as it requires only a single simple push-button type of movement to open the container.

Other features and advantages are described below. The invention resides as well in subcombinations of the features shown and described.

### BRIEF DESCRIPTION OF THE DRAWINGS

In the drawings, wherein the same reference number indicates the same element in each of the views:

FIG. 1 is a rear perspective view of a unit dose dry powder inhaler.

FIG. 2 is a front and side perspective view of the inhaler shown in FIG. 1.

FIG. 3 is a front view of the inhaler shown in FIG. 1, in a packaged or storage position.

FIG. 4 is a front view of the inhaler of FIG. 1, in a ready for use condition.

FIG. 5 is a perspective view of the mouthpiece plate and dose unit shown in FIGS. 1 and 2.

FIG. 6 is a perspective view similar to FIG. 5, with the dose unit removed for clarity of illustration.

FIG. 7 is a perspective view of the base plate shown in FIG. 1, and also showing the reverse side of the dose unit shown in FIG. 5.

FIG. 8 is a perspective view similar to FIG. 7, with the dose unit removed for clarity of illustration.

FIG. 9 is a schematic illustration of the inhaler as shown in FIG. 3.

FIG. 10 is a schematic illustration of the inhaler as shown in FIG. 4.

FIG. 11 is a bottom view of the dose unit shown in FIGS. 5, 7, 9 and 10.

### DETAILED DESCRIPTION OF THE DRAWINGS

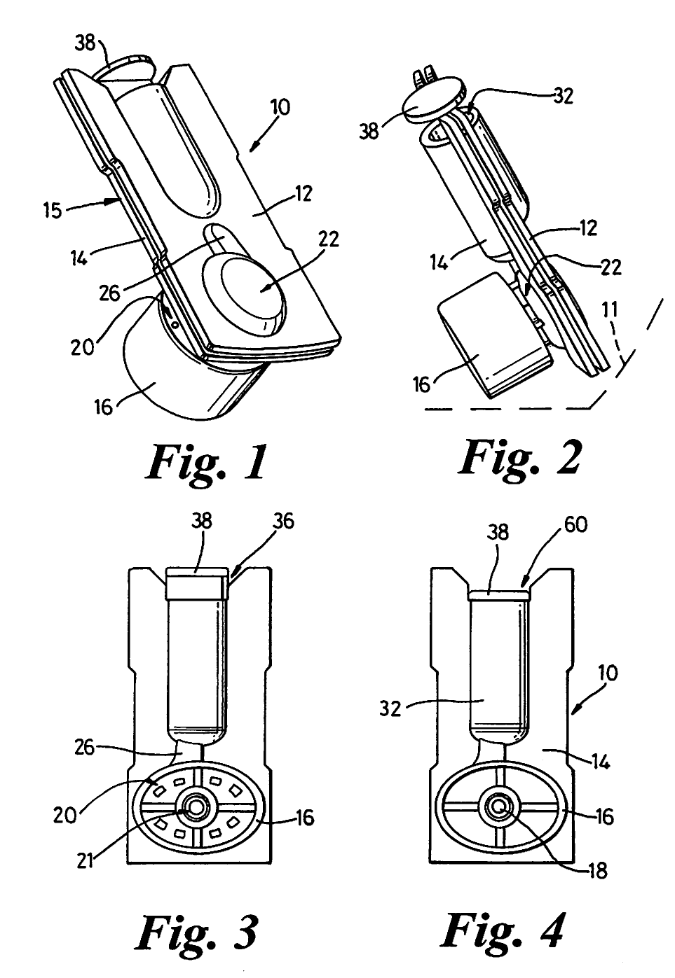

As shown in FIGS. 1-4, a unit dose dry powder inhaler 10 has a base or bottom plate 12 attached to a top or mouthpiece plate 14, to form an inhaler housing 15.

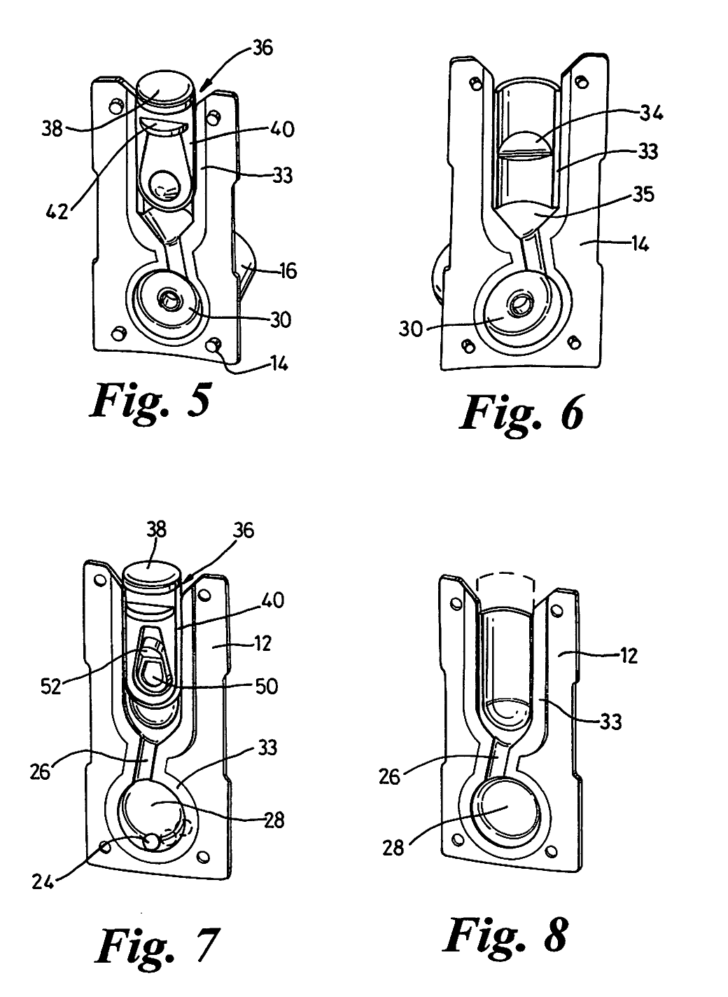

Referring now also to FIGS. 5-8, a dispersion chamber 22 is formed within the housing 15, by a base chamber wall or section 28 on the base plate 12, together with a top chamber wall or section 30 on the top plate 14. One or more beads 24 are contained within the dispersion chamber 22, as described in U.S. Pat. No. 6,427,688, and U.S. Published Patent Application No. 2001/0027790A1, both incorporated herein by reference. A cylindrical dose tube 32 is formed within the housing 15. A tapered or conical area 35 is optionally provided at the inner end of the dose tube 32. A chamber inlet passageway or tube 26 extends between and connects the dose tube 32 into the dispersion chamber 22. A raised rim 33 may be provided on one or both of the base plate 12 and the top plate 14 surrounding the dose tube 32, the chamber inlet passageway 26, and/or the dispersion chamber 22, for ease of manufacture, or to help confine air flow within these spaces, or both.

Referring to FIGS. 1, 3 and 4, a chamber tube 18 extends from the dispersion chamber 22 up through the mouthpiece 16. Sheath air openings 20 are optionally provided in the mouthpiece 20. A toroidal sheath air opening 21 may also be provided surrounding the chamber tube 18.

Turning now to FIGS. 5, 7 and 9-11, a dose unit, generally designated 36, has an end cap or button 38 on a plate 40. An alignment ring or sector 42 extends outwardly from the plate 40, similar to the cap 38. A tab 48 is positioned within a tab slot or opening 58 in the plate 40. The tab 48 is supported on a pair of pivot pins or supports 56, allowing the tab 48 to pivot relative to the plate 40 under nominal force. A second or lever section 52 of the tab 48 is joined to a first or blister section 50 of the tab 48 at an obtuse angle, e.g., 100-160 degrees, 125-145 degrees, or about 130 degrees. The intersection of the lever section 52 and the blister section 50 of the tab 48 is advantageously located at or adjacent to the pivot axis 54 extending through the pivot pins 56.

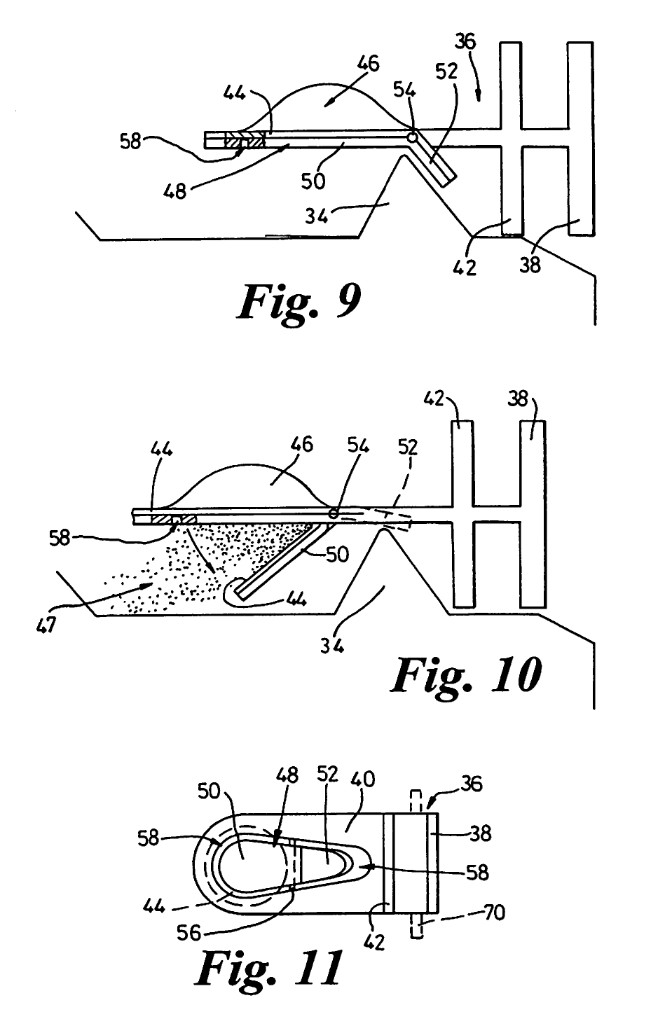

Referring to FIGS. 9-11, a blister 46 containing a dry powder 47 is provided on the tab 48. The blister 46 includes a conical or domed top surface attached to a flat seal layer 44. The seal layer 44, typically a metal foil, is adhered or otherwise attached onto the tab 48, as described in U.S. Pat. No. 5,622,166 and U.S. Pat. Des. 384,283, both incorporated herein by reference. The central area of the seal layer 44 is attached to the blister section 50 of the tab 48, with the seal layer 44 of the blister 46 also overlying the tab slot or opening 58 and attached to the plate 40, as shown in dotted lines in FIG. 11.

In use, the inhaler 10 is typically provided within an envelope or package 11 in an assembled and ready to use condition. A dose unit assembly 36 is preinstalled within the chamber tube 18 of the housing 15, as shown in FIG. 3. The lever section 52 of the tab 48 is adjacent to, or in light contact with, the tab ramp 34, as shown in FIGS. 5-7 and 9. When the user is ready to inhale a dose of medicine 47, the user pushes on the end cap 38 of the dose unit 36. The dose unit 36 slides within the chamber tube 18, from the position shown in FIGS. 3 and 9, to the position shown in FIGS. 4 and 10. As this occurs the lever section 52 moves against the tab ramp 34. The tab 48 pivots about the pivot line 54, or about the pivot pins 56. The seal layer 44 is sheared out from the bottom of the blister 46, as the blister section 50 of the tab 48 pivots downwardly. This movement releases the powder 47 into the inside or front end of the chamber tube 18.

The user then places the mouthpiece 16 into the mouth and inhales. Air flows through the chamber tube 18 entraining and carrying the powder 47 through the chamber inlet 26 and into the dispersion chamber 22. The beads 24 in the dispersion chamber 22 move rapidly within the chamber, helping to disperse the powder 47. The dispersed powder/air mixture flows out of the dispersion chamber 22 into the chamber tube 18, and into the user's lungs. Sheath air is optionally provided via sheath air passageways 20 or 21, to help reduce deposition of powder particles on or in the mouthpiece 16, or the user's mouth and throat. Improved deposition of powder 47 into the user's deep lung is achieved.

As shown in FIGS. 4 and 10, a cap recess 60 may be provided in the housing 15, to reduce the potential for inadvertent pressing of the dose unit into the dose tube, during packaging, shipping, storage or handling. A detent or latching or locking device may optionally be provided on the dose unit 36, to prevent its removal from the housing 15, either before use, in the position shown in FIG. 9, or after use, in the position shown in FIG. 10. Alternatively, for certain medicines or dosing regimens, the dose unit may be dimensioned so that it can be easily pulled out of the housing, removed, and replaced with a new dose unit. This allows for repeated use of the inhaler.

The dose unit 36 is preferably molded of a plastics material, for ease of manufacture, and with the pivot pins 56 molded in place.

The alignment ring or sector 42 helps to center and align the dose unit 36 within the chamber tube 18. Keying features may also be provided on the dose unit 36, to ensure proper angular alignment of the dose unit 36 within the chamber tube 18, so that the lever section 52 of the tab 48 properly aligns with and contacts the tab ramp 34 during use. As shown in dotted lines in FIG. 11, the dose unit 36 may include one or more wings 70 which are permanently broken, deflected, bent, or sheared off, when the dose unit 36 is pressed into the dose tube 32. If the used dose unit is removed from the dose tube 32, the wings 70 provide a visual indication that the dose unit is used and should be discarded. The potential for inadvertent attempts to use an already used and empty dose unit are therefore reduced.

Although the housing 15 is shown as a two-piece construction including the base plate 12 and the top plate 14, single piece or multiple piece housing designs are also feasible. In addition, while the housing 15 is described here as formed by plates 12 and 14, the housing 15 may of course have other shapes and construction characteristics. Similarly, other types of dispersion chambers, with or without beads 24, may be used. For example, dispersion chambers 22 having baffles, deflection surfaces, impellers, etc., may be substituted for the dispersion chamber 22 described above. Similarly, various mouthpiece designs and configurations may also be used. While the mouthpiece 16 and chamber tube 18 are shown in the drawings as perpendicular to the chamber tube 18, parallel/flow-through designs or other positional relationships between them may be used. These features described immediately above are not essential elements of the invention.

The specific size, shape and arrangement of various of the features shown FIGS. 1 and 2 is arbitrary. The appearance of these features is a matter of design preference from a large array of options. These features may be selected or changed to create any desired ornamental external appearance of the inhaler 10.

Thus, a novel inhaler has been shown and described. Various changes and substitutions may of course be made, without departing from the spirit and scope of the invention. The invention, therefore, should not be limited, except to the following claims, and their equivalents.

1. A single dose dry powder inhaler, comprising:
a housing;
a powder dispersion chamber within the housing;
a mouthpiece on the housing connecting with the dispersion chamber;
a dose unit insertable into the housing, for delivering a single dose into the dispersion chamber, with the dose unit comprising:
a frame;
a tab pivotably attached to the frame, and with the tab including a first tab section, and a second tab section joined at a fixed angle to the first tab section, and a dose container on the tab, containing a single dose of dry powder;
with the dose unit moveable from a first position wherein the dose container remains closed, to a second position, wherein the dose container is opened.

2. The inhaler of claim 1 with the second tab section joined to the first tab section at a fixed obtuse angle.

3. The inhaler of claim 1 with the tab including the first and second tab sections formed as a single plastic molded component.

4. The inhaler of claim 1 wherein the dose container comprises a blister having a metal foil bottom adhered to the first tab and to the second section.

5. The inhaler of claim 1 further comprising a tab slot substantially surrounding the tab, and with the tab pivotably supported within the tab slot by pivot supports extending across the tab slot.

6. The inhaler of claim 1 further comprising a ramp in the housing, and with ramp contacting the second tab section, as the dose unit is moved linearly in the housing, to pivot the tab and open the dose container.

7. The inhaler of claim 1 further comprising an end cap on the dose unit generally aligned with an edge of the housing, when the dose unit is in the first position, and recessed below the edge of the housing when the dose unit is in the second position.

8. The inhaler of claim 1 with the housing comprising a base or bottom plate joined with a mouthpiece or top plate.

9. The inhaler of claim 1 wherein the dose unit is moveable linearly within the housing along a first axis, from the first position to the second position, and wherein the mouthpiece has an outlet opening leading into the dispersion chamber, along a second axis, and wherein the second axis is perpendicular to the first axis.

10. The inhaler of claim 3 further comprising an alignment sector on the dose unit adapted to keep the dose unit aligned in the chamber tube within the housing.

11. The inhaler of claim 3 further comprising a chamber inlet extending from the chamber tube to the dispersion chamber.

12. A dry powder inhaler, comprising:
a housing;
a powder dispersion chamber within the housing;
a mouthpiece on the housing connecting with the dispersion chamber;
a dose unit insertable into the housing, for delivering a single dose into the dispersion chamber, with the dose unit including a sealed powder container; and
container opening means for opening the sealed container, by linearly moving the dose unit, within the housing from a first position wherein the dose container remains closed, to a second position, wherein the dose container is opened.

13. The inhaler of claim 12 wherein the container opening means comprises a dose unit having a tab pivotably attached to a frame, and with the tab parallel to the frame when the dose unit is in the first position, and with the tab pivoted to a non parallel position, when the dose unit is moved via the user's finger force into the second position.

14. A dose unit for providing a single dose of a dry powder pharmaceutical, comprising:
a frame having a tab opening;
a tab in the tab opening and including a first tab section, and a second tab section joined at an angle to the first tab section;
a pivot attachment pivotably attaching the tab to the frame; and
a dose container on the frame and the first tab section, and containing a single dose of dry powder.

15. The dose unit of claim 14 with the dose container having a bottom foil layer adhered to both the frame and to the first section, with the bottom foil layer shearable with pivoting movement of the tab, to release the powder from the dose container.

16. The dose unit of claim 14 wherein the angle is a fixed obtuse angle.

17. The dose unit of claim 14 further comprising an end cap on the frame.

18. The dose unit of claim 14 further comprising an alignment ring or sector on the frame.

19. The dose unit of claim 14 wherein the frame is flat, the first tab section is parallel to the frame, and the second tab section extends out of the plane of the frame.

20. The dose unit of claim 14 wherein the first and second tab sections comprise a single plastic molded component.
